(19) United States
(12) Patent Application Publication
(10) Pub. No.: US 2012/0246757 A1
(43) Pub. Date: Sep. 27, 2012

(54) MODULATION OF CELLULAR PROTEIN FUNCTION BY ARTIFICIAL SUMO LIGASES

(75) Inventors: German Rosas-Acosta, El Paso, TX (US); Sangita Pal, Houston, TX (US)

(73) Assignee: BOARD OF REGENTS, THE UNIVERSITY OF TEXAS SYSTEM, Austin, TX (US)

(21) Appl. No.: 13/427,046

(22) Filed: Mar. 22, 2012

Related U.S. Application Data
(60) Provisional application No. 61/467,886, filed on Mar. 25, 2011.

(51) Int. Cl. A61K 38/53; C12N 15/79; C12N 5/071; A01H 1/06; C12Q 1/70; G01N 33/564; A61P 25/28; C12N 9/96; C12N 5/04

(52) U.S. Cl. 800/278; 435/188; 435/440; 435/375; 435/410; 435/5; 435/7.6; 424/94.3

(57) ABSTRACT

A development of an Artificial SUMO Ligase (ASUL) to increase the ability of Ubc9 to interact with the SUMO target protein, therefore increasing the rate of SUMOylation of the target and a net increase in the total amount of SUMOylated target protein in the cell is described herein. The method of the present invention involves the creation of a protein fusion between a protein domain known to interact with the target protein to be SUMOylated (ID) and the SUMO conjugating enzyme Ubc9. Compositions and methods involving an ASUL comprising a fusion of the N-terminal domain of influenza A virus non-structural protein (NS1) and Ubc9 is also described.

### CROSS-REFERENCE TO RELATED APPLICATIONS

This application claims priority based on U.S. provisional Application No. 61/467,886, filed Mar. 25, 2011. The contents of which is incorporated by reference in its entirety.

### STATEMENT OF FEDERALLY FUNDED RESEARCH

This invention was made with U.S. Government support under Contract No. 1SC2AI081377-02 awarded by the NIH/NIAID and NIGMS. The government has certain rights in this invention.

### TECHNICAL FIELD OF THE INVENTION

The present invention relates in general to the field of protein function regulation, and more specifically to SUMO (Small Ubiquitin-like MOdifier)-mediated regulation of protein function by the specific alteration of properties of the target protein.

### REFERENCE TO A SEQUENCE LISTING

None.

### BACKGROUND OF THE INVENTION

Without limiting the scope of the invention, its background is described in connection with protein function regulation.

U.S. Pat. No. 7,750,134 issued to Godzik and Reed (2010) provides isolated SUMO-specific protease-like (or “SSP”) domain-containing polypeptides from microorganisms, including bacteria, protozoans and yeast, including Escherichia, Salmonella, Pseudomonas, Chlamydia, Plasmodium, Trypanosma, Mesorhizobium, Rickettsia, Cryptosporidium and Candida species. The invention further provides modifications of such polypeptides, functional fragments therefrom, encoding nucleic acid molecules and specific antibodies. Also provided are methods for identifying polypeptides and compounds that associate with or modulate the activity of the SSP domain-containing polypeptides. Further provided are methods of modulating a biological activity in a cell, and treating pathological conditions, using the described nucleic acid molecules, polypeptides and compounds

### SUMMARY OF THE INVENTION

The present invention describes a method of increasing the rate of SUMOylation of the target and a net increase in the total amount of SUMOylated target protein in the cell by the development of an artificial SUMO ligase (ASUL) and compositions and uses thereof are described in various embodiments of the present invention.

The present invention in one embodiment discloses a fusion protein for increasing a rate, a specificity or both of Small Ubiquitin-like Modifier attachment (SUMOylation) to a target protein comprising: (i) an interaction domain (ID) comprising a protein, a protein fragment, a peptide or combinations and modifications thereof, wherein the ID capable of interacting or binding to the target protein, a region or a site of the target protein undergoing the SUMOylation, (ii) one or more conjugating enzymes or enzyme complexes, wherein the conjugating enzymes catalyze, modulate or promote the SUMOylation in the target protein, (iii) a Small Ubiquitin-like Modifier (SUMO) attached to the enzyme or the enzyme complex, and a spacer, a linker or a hinge region connecting the ID with the one or more enzymes or enzyme complexes. The target protein as disclosed hereinabove is a bacterial protein, a viral protein, a plant protein, an animal protein, a human protein, or combinations thereof. In one aspect of the conjugating enzyme comprises an ubiquitin-conjugating enzyme, more specifically the ubiquitin-conjugating enzyme is Ubc9. In another aspect the fusion protein of the present invention is activated by one or more SUMO activating enzymes, wherein the SUMO activating enzyme is E1 SUMO activating enzyme (SAE2/1).

In yet another aspect, the enzyme or the enzyme complex may optionally comprise one or more SUMO activating enzymes (E1), SUMO ligases (E3) or a combination. In another aspect the one or more SUMO ligases are selected from the group consisting of protein inhibitor of activated Stat proteins (PIAS) comprising PIAS1 and PIASxβ, protein inhibitor of activated STAT proteins comprising Siz1 and Siz2/Nfi1, and Mms21. In one aspect of the fusion protein disclosed above a length of the spacer is determined by both a size of the ID and a distance of the ID from a SUMOylation site in the target protein. In another aspect the spacer orients the one or more enzymes or enzyme complexes with respect to the target protein or the SUMOylation site in the target protein.

Another embodiment of the present invention relates to a fusion protein (NS1₁₋₈₇-Ubc9) for increasing a rate, a specificity or both of Small Ubiquitin-like Modifier attachment (SUMOylation) to an influenza A virus non-structural protein (NS1) comprising: (i) an interaction domain (ID) comprising amino acids 1-87 from a N-terminal region of the NS1 protein, (ii) an ubiquitin-conjugating enzyme, wherein the ubiquitin-conjugating enzyme is Ubc9, (iii) a Small Ubiquitin-like Modifier (SUMO) attached to the Ubc9, and (iv) a spacer, a linker or a hinge region connecting the ID with the Ubc9. In one aspect the fusion protein is activated by one or more SUMO activating enzymes. In another aspect the SUMO activating enzyme is E1 SUMO activating enzyme (SAE2/1). In yet another aspect a length of the spacer is determined by both a size of the ID and a distance of the ID from a SUMOylation site in the NS1 protein. In another aspect the spacer orients the Ubc9 with respect to the NS1 protein or the SUMOylation site in the NS1 protein.

In yet another embodiment, the present invention provides a method for increasing a rate, a specificity or both of Small Ubiquitin-like Modifier attachment (SUMOylation) to a target protein in a human or an animal subject, a plant or a cell comprising the step of injecting, introducing or transfecting a fusion protein or one or more plasmids expressing the fusion protein. The fusion protein as described in the method of the present invention comprises: i) an interaction domain (ID) comprising a protein, a protein fragment, a peptide or combinations and modifications thereof, wherein the ID capable of interacting or binding to the target protein, a region or a site of the target protein undergoing the SUMOylation, ii) one or more conjugating enzymes or enzyme complexes, wherein the conjugating enzymes catalyze, modulate or promote the SUMOylation in the target protein, iii) a Small Ubiquitin-like Modifier (SUMO) attached to the enzyme or the enzyme complex, and iv) a spacer, a linker or a hinge region connecting the ID with the one or more enzymes or enzyme complexes.

In one aspect, the method further comprises the step of measuring a level of SUMOylation, an increase in a level of a SUMOylated target protein or both in the human or the animal subject, the plant or the cell prior to and after the injection, introduction or transfection of the fusion protein or the one or more expression plasmids. In another aspect the conjugating enzyme comprises an ubiquitin-conjugating enzyme, wherein the ubiquitin-conjugating enzyme is Ubc9. In yet another aspect the fusion protein is activated by one or more SUMO activating enzymes, wherein the SUMO activating enzyme is E1 SUMO activating enzyme (SAE2/1).

The enzyme or the enzyme complex disclosed in the method hereinabove may optionally comprise one or more SUMO activating enzymes (E1), SUMO ligases (E3), or a combination, wherein the one or more SUMO ligases are selected from the group consisting of protein inhibitor of activated Stat proteins (PIAS) comprising PIAS1 and PIASxβ, protein inhibitor of activated STAT proteins comprising Siz1 and Siz2/Nfi1, and Mms21. In another aspect a length of the spacer is determined by both a size of the ID and a distance of the ID from a SUMOylation site in the target protein. In yet another aspect the spacer orients the one or more enzymes or enzyme complexes with respect to the target protein or the SUMOylation site in the target protein.

The present invention further discloses a method for increasing a rate, a specificity or both of Small Ubiquitin-like Modifier attachment (SUMOylation) to an influenza A virus non-structural protein (NS1) in a human or animal subject or a cell comprising the steps of injecting, introducing or transfecting a fusion protein (NS11-87-Ubc9), one or more plasmids expressing the fusion protein, wherein the fusion protein comprises: a) an interaction domain (ID) comprising amino acids 1-87 from a N-terminal region of the NS1 protein; b) an ubiquitin-conjugating enzyme, wherein the ubiquitin-conjugating enzyme is Ubc9, c) a Small Ubiquitin-like Modifier (SUMO) attached to the Ubc9, and d) a spacer, a linker or a hinge region connecting the ID with the Ubc9. In one aspect, the method further comprises the step of measuring a level of SUMOylation, an increase in a level of a SUMOylated NS1 protein or both in the human or animal subject or the cell prior to and after the introduction or transfection of the one or more expression plasmids. In another aspect the fusion protein is activated by one or more SUMO activating enzymes. In yet another aspect, the SUMO activating enzyme is E1 SUMO activating enzyme (SAE2/1). In another aspect of the method the length of the spacer is determined by both a size of the ID and a distance of the ID from a SUMOylation site in the NS1 protein. In a related aspect the spacer orients the Ubc9 with respect to the NS1 protein or the SUMOylation site in the NS1 protein.

The present invention in one embodiment details a method of characterizing a target protein, studying a structure, a function or any combinations thereof comprising the steps of: (i) promoting, increasing a rate, a specificity or combinations thereof of Small Ubiquitin-like Modifier attachment (SUMOylation) to the target protein in a human or animal subject, a plant or a cell, (ii) providing a fusion protein comprising: a) an interaction domain (ID) comprising a protein, a protein fragment, a peptide or combinations and modifications thereof, wherein the ID capable of interacting or binding to the target protein, a region or a site of the target protein undergoing the SUMOylation; b) one or more conjugating enzymes or enzyme complexes, wherein the conjugating enzymes catalyze, modulate or promote the SUMOylation in the target protein; c) a Small Ubiquitin-like Modifier (SUMO) attached to the enzyme or the enzyme complex; and d) a spacer, a linker or a hinge region connecting the ID with the one or more enzymes or enzyme complexes, (iii) introducing or transfecting the fusion protein or one or more plasmids expressing the fusion protein in the human or animal subject, the plant or the cell, (iv) measuring a level of SUMOylation, an increase in a level of a SUMOylated target protein or both in the human or animal subject, the plant or the cell; and (v) characterizing the SUMOylated target protein, studying the structure, the function or any combinations thereof by using one or more analytical, structural or functional techniques.

In one aspect of the method, the conjugating enzyme comprises an ubiquitin-conjugating enzyme, wherein the ubiquitin-conjugating enzyme is Ubc9. In another aspect, the fusion protein is activated by one or more SUMO activating enzymes, wherein the SUMO activating enzyme is E1 SUMO activating enzyme (SAE2/1). In yet another aspect, the enzyme or the enzyme complex may optionally comprise one or more SUMO activating enzymes (E1), SUMO ligases (E3) or a combination. In another aspect, a length of the spacer is determined by both a size of the ID and a distance of the ID from a SUMOylation site in the target protein. In a related aspect the spacer orients the one or more enzymes or enzyme complexes with respect to the target protein or the SUMOylation site in the target protein. In yet another aspect, the method requires an a priori knowledge of a localization of the SUMOylation site and of specific target protein interacting partners.

Another embodiment of the instant invention relates to a method for treating Alzheimer's disease (AD) in a human subject by increasing a rate, a specificity or combinations thereof of Small Ubiquitin-like Modifier attachment (SUMOylation) to one or more amyloid precursor proteins (APP), wherein the SUMOylation increases a solubility or decreasing an aggregation or both, comprising the steps of: (i) identifying the subject in need for treatment against the AD; and (ii) administering a therapeutically effective amount of a fusion protein comprising: a) an interaction domain (ID) comprising a protein, a protein fragment, a peptide or combinations and modifications thereof, wherein the ID capable of interacting or binding to the APP, a region or a site of the APP undergoing the SUMOylation; b) one or more conjugating enzymes or enzyme complexes, wherein the conjugating enzymes catalyze, modulate or promote the SUMOylation in the APP; c) a Small Ubiquitin-like Modifier (SUMO) attached to the enzyme or the enzyme complex; and d) a spacer, a linker or a hinge region connecting the ID with the one or more enzymes or enzyme complexes.

In one aspect, the instant invention further comprises the step of monitoring a progression of the treatment by measuring a change in the solubility, the aggregation or both of the one or more APP prior to or after the administration of the fusion protein. In another aspect the conjugating enzyme comprises an ubiquitin-conjugating enzyme, wherein the ubiquitin-conjugating enzyme is Ubc9. In yet another aspect, the fusion protein is activated by one or more SUMO activating enzymes, wherein the SUMO activating enzyme is E1 SUMO activating enzyme (SAE2/1). In a related aspect the enzyme or the enzyme complex may optionally comprise one or more SUMO activating enzymes (E1), SUMO ligases (E3) or a combination. In another aspect, the fusion protein is administered parenterally.

In yet another embodiment, the instant invention provides a method for treating one or more diseases associated with defects in the Small Ubiquitin-like Modifier attachment SUMOylation of specific cellular proteins in a human subject by increasing a rate, a specificity or combinations thereof of (SUMOylation) to one or more cellular proteins comprising the steps of: (i) identifying the subject in need for treatment against the one or more diseases; and (ii) administering a therapeutically effective amount of a fusion protein comprising: an interaction domain (ID) comprising a protein, a protein fragment, a peptide or combinations and modifications thereof, wherein the ID capable of interacting or binding to the cellular protein, a region or a site of the cellular protein undergoing the SUMOylation, one or more conjugating enzymes or enzyme complexes, wherein the conjugating enzymes catalyze, modulate or promote the SUMOylation in the cellular protein, a Small Ubiquitin-like Modifier (SUMO) attached to the enzyme or the enzyme complex, and a spacer, a linker or a hinge region connecting the ID with the one or more enzymes or enzyme complexes.

The method as described hereinabove, further comprises the step of monitoring a progression of the treatment by measuring a level of SUMOylation, an increase in a level of a SUMOylated cellular protein or both in the human subject prior to and after the administration of the fusion protein. In specific aspects, the disease is familial dilated cardiomyopathy and the cellular protein is Lamin A. In one aspect, the conjugating enzyme comprises an ubiquitin-conjugating enzyme, wherein the ubiquitin-conjugating enzyme is Ubc9. In another aspect, the fusion protein is activated by one or more SUMO activating enzymes, wherein the SUMO activating enzyme is E1 SUMO activating enzyme (SAE2/1). In yet another aspect, the enzyme or the enzyme complex may optionally comprise one or more SUMO activating enzymes (E1), SUMO ligases (E3) or a combination. In a related aspect, the fusion protein is administered parenterally.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic showing the balance between SUMO conjugation and deconjugation;

FIG. 2 shows the development of an artificial SUMO ligase (ASUL) according to an embodiment of the present invention;

FIG. 3 is a schematic representation showing an increased SUMOylation mediated by an artificial SUMO ligase (ASUL) according to an embodiment of the present invention;

FIGS. 4A-4D shows that the fusion proteins NS1₁₋₈₇-Ubc9 act as an effective Artificial SUMO ligase (ASUL) for full-length NS1:

FIGS. 5A-5E shows that the ASUL activity mediated by NS1₁₋₈₇-Ubc9 is specific, i.e., that the ligase activity mediated by NS1₁₋₈₇-Ubc9 does not result in a global increase in cellular protein SUMOylation and that NS1₁₋₈₇-Ubc9 does not enhance the SUMOylation of a mutant form of the target protein lacking the specific lysine residues normally targeted by the SUMOylation system.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

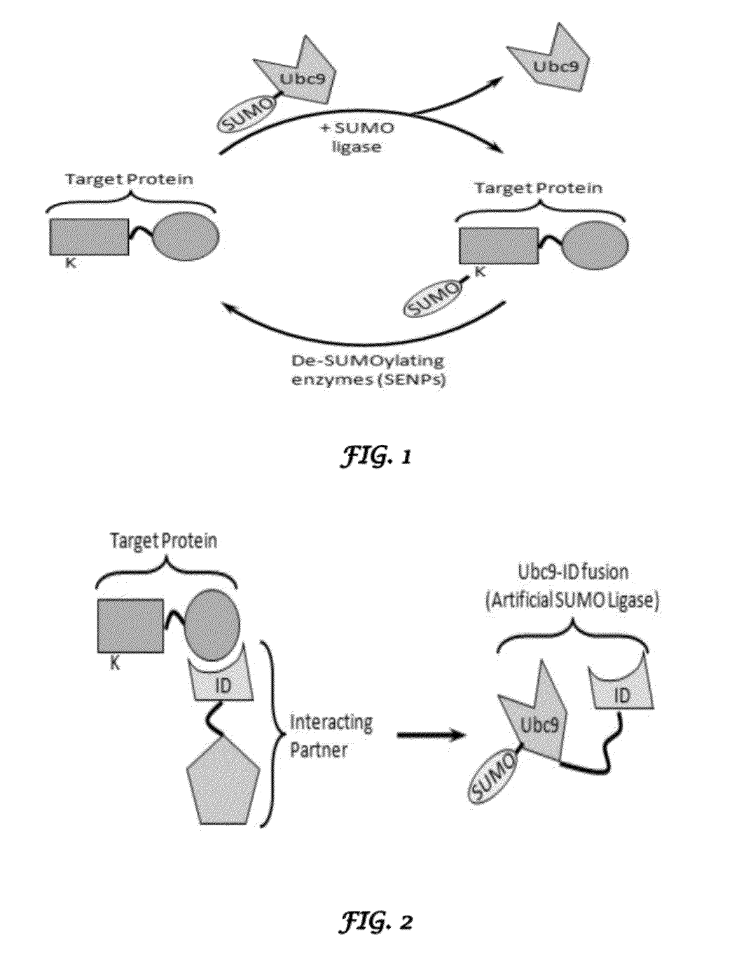

SUMOylation is a post-translational modification involving the formation of an isopeptide link between the C-terminus of SUMO and the epsilon amino group of a Lysine residue located in the target protein. SUMOylation is achieved by the concerted action of the SUMO conjugating enzyme Ubc9 and SUMO ligases that provide speed and specificity to the SUMO conjugation process. Most SUMOylated proteins are rapidly de-SUMOylated due to the activity of the cellular de-SUMOylating enzymes, known as SENPs (FIG. 1). SUMOylation is known to regulate protein function by affecting, in a protein-specific manner, the stability, cellular localization, and molecular interactions established by the target protein¹⁻³.

Most studies aimed at characterizing the effects of SUMO on its targets rely on mapping the SUMOylation site on the SUMO target, followed by a comparison of the activities associated to the SUMOylatable form of the protein versus those of a non-SUMOylatable form, in which the Lysine residues targeted by the SUMOylation system are substituted by either Arginine or Alanine Although substantial progress has been achieved the understanding of the activities associated to the cellular SUMOylation system, it would be desirable to have the ability to specifically increase the SUMOylation of the SUMO target under study. Currently, the only available methodology to this end, the so-called Ubc9-fusion directed SUMOylation or UFDS methodology, requires modifying the protein target itself by fusing it to the SUMOylating enzyme Ubc9⁴⁻⁶. Although such fusion dramatically increases the SUMOylation of the target protein under study, it is likely that it may also alter the properties of the target protein in unpredictable ways, therefore producing effects not related to SUMO conjugation. Furthermore, the need to produce a fusion between the target and Ubc9 forces the studies to be executed by over-expression of an exogenously added copy of the gene, instead of using the endogenously expressed protein. An unquestionable proof of the many limitations associated to the use of the UFDS methodology is the fact that since its initial description in the literature in 2007, this method has been cited only 14 times, mostly while being compared to other methods, and it has been used in the analysis of a very limited number of SUMO targets.

The development of an Artificial SUMO Ligase (ASUL) involves the creation of a protein fusion between a protein domain known to interact with the target protein to be SUMOylated (hereafter referred to as the “Interaction Domain” or ID) and the SUMO conjugating enzyme Ubc9 (FIG. 2). The type of fusion to be created depends on the location of the SUMOylation site in the target protein in relation with the region known to interact with the protein domain to be fused to Ubc9. The goal is to design an ID-Ubc9 fusion that will place Ubc9 in close proximity with the target SUMOylation site. This may require the creation of either C- or N-terminal Ubc9 fusions with the ID. A spacer or hinge region must be included between the ID and Ubc9 to provide spatial mobility to Ubc9 so that it will have the freedom required to position itself in the appropriate orientation to SUMOylate the desired Lysine residue in the target protein. The length of the spacer must be determined empirically and depends on both, the distance between the ID and the SUMOylation site on the target protein, and the size of the ID.

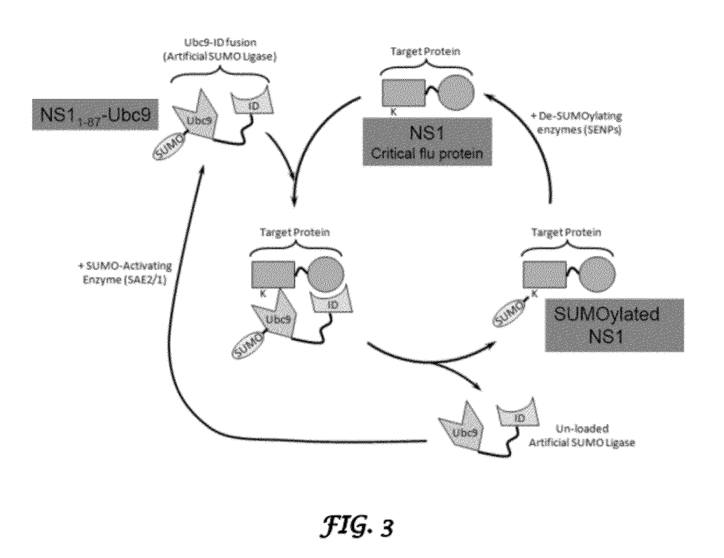

The fusion of the ID and Ubc9 increases the ability of Ubc9 to interact with the SUMO target protein (FIG. 3), therefore increasing the rate of SUMOylation of the target. This in turn results in a net increase in the total amount of SUMOylated target protein in the cell. Therefore, to be fully functional and successfully increase the SUMOylation of the target protein under study, the Ubc9-ID fusion must be normally activated by the E1 SUMO activating enzyme (SAE2/1), and must be able to interact with the target protein and position Ubc9 in close proximity with the SUMOylation site, thus acting as an adaptor. This latter activity corresponds to the one postulated to be exerted by some of the known SUMO ligases, such as the members of the PIAS family of SUMO ligases⁸.

To demonstrate the applicability of the idea described above, the inventors increased the SUMOylation of the influenza A virus non-structural protein NS1 by developing an ASUL specific for this viral protein. NS1 was recently characterized as an authentic SUMO target by the present inventors⁹. NS1 exhibits two main domains, an N-terminal domain endowed with RNA binding ability and responsible for the formation of NS1 homodimers, and a C-terminal domain known as the “effector domain”, which confers to NS1 most of its known activities. These two domains are separated by a flexible hinge region of 15 amino acid residues (residues 71-85).

Considering the known ability of NS1A to form dimers mediated by its N-terminal RNA binding domain, and the similar size of Ubc9 and the C-terminal domain of NS1, the inventors hypothesize that a fusion of the N-terminal domain of NS1 and Ubc9 would likely act as an effective ASUL to increase the SUMOylation of full-length NS1. Thus, the inventors introduced Ubc9's ORF downstream of the sequence coding for amino acid residues 1-87 of NS1. This region of NS1 comprises the N-terminal dimerization/RNA binding domain and the hinge region.

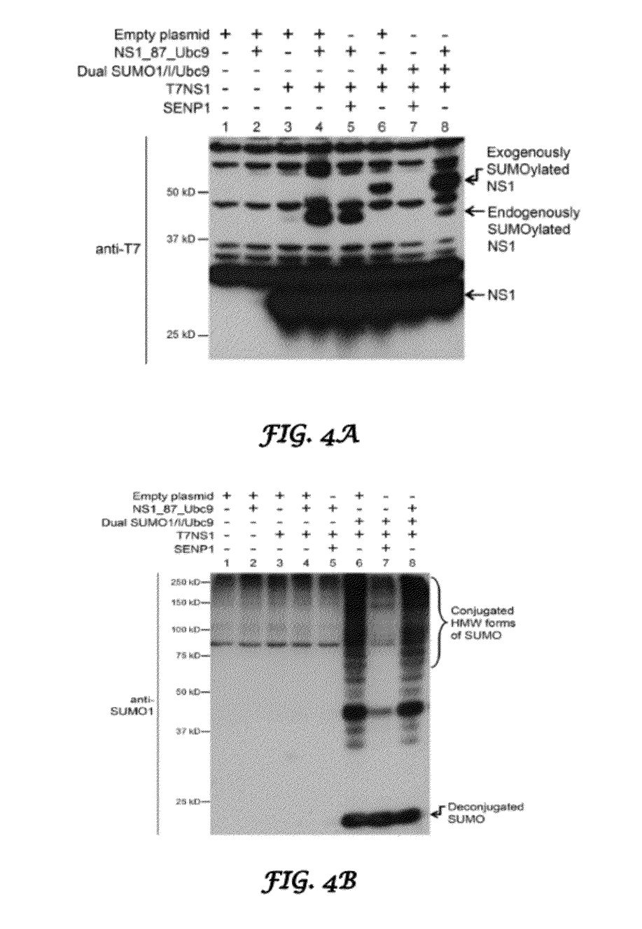

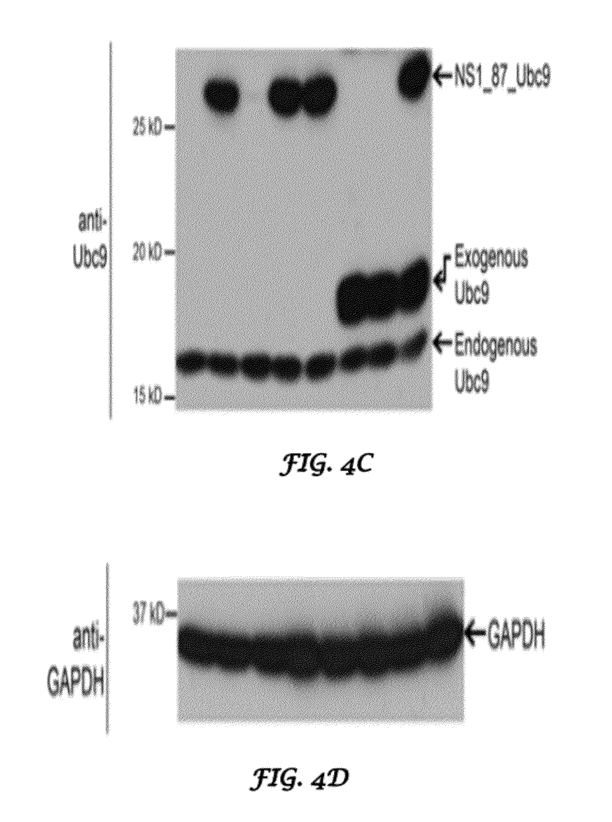

To test the SUMO ligase activity of the fusion protein produced, henceforth referred to as NS1₁₋₈₇-Ubc9, the inventors co-transfected HEK293A cells with various combinations of an empty plasmid control, the dicistronic expression plasmid Dual SUMO1/I/Ubc9 coding for SUMO1 and Ubc9 and previously shown to produce a dramatic up-regulation of global SUMOylation⁹, an expression plasmid for a T7-tagged full-length NS1, an expression plasmid for the de-SUMOylating enzyme SENP1, and the expression plasmid for NS1₁₋₈₇-Ubc9. FIGS. 4A-4D shows that the fusion proteins NS1₁₋₈₇-Ubc9 act as an effective Artificial SUMO ligase (ASUL) for full-length NS1. Over-expression of NS1 by itself led to almost undetectable levels of NS1 SUMOylation (FIG. 4A, lane 3). However, when NS1 was over-expressed together with NS1₁₋₈₇-Ubc9, its SUMOylation was dramatically increased (FIG. 4A, lane 4), but the global levels of cellular SUMOylation appeared unaffected (FIG. 4B, lane 4). Furthermore, co-expression of the de-SUMOylating enzyme SENP1 in the presence of NS1₁₋₈₇-Ubc9 decreased NS1 SUMOylation but did not preclude it completely (FIG. 4A, lane 5). In contrast, co-transfection with Dual SUMO1/I/Ubc9 produced a substantial increase in NS1 SUMOylation (FIG. 4A, lane 6), albeit slightly less dramatic than the one produced by co-expression of NS1₁₋₈₇-Ubc9, but such increase was accompanied by a global increase in cellular SUMOylation (FIG. 4B, lane 6), and was completely abolished by co-expression of SENP1 (FIG. 4A, lane 7), which also decreased global cellular SUMOylation (FIG. 4B, lane 7). Co-transfection with Dual SUMO1/I/Ubc9 and NS1₁₋₈₇-Ubc9 led to the most dramatic increase in NS1 SUMOylation (FIG. 4A, lane 8), but also produced the most dramatic increase in global cellular SUMOylation (FIG. 4B, lane 8). The anti-Ubc9 immunoblot analysis confirmed the expression of the NS1₁₋₈₇-Ubc9 fusion protein in the corresponding samples and the anti-GAPDH immunoblot analysis demonstrated that all lanes contained equivalent amounts of protein. Altogether, the data described above demonstrates that NS1₁₋₈₇-Ubc9 works as an effective Artificial SUMO Ligase (ASUL) for full-length NS1, producing a highly specific increase in the SUMOylation of NS1.

To further demonstrate the specificity of the NS1₁₋₈₇-Ubc9 construct the inventors tested whether the increased SUMOylation previously observed mediated by its ASUL activity would be affected by mutating the previously mapped SUMOylation sites in NS1, i.e., residues K70 (secondary SUMOylation site) and K219 (primary SUMOylation site). To this end, the inventors co-transfected HEK293A with various combinations of an empty plasmid control, the NS1₁₋₈₇-Ubc9 construct, SENP1, and T7-tagged NS1 expression plasmids, including two containing specific Lysine to Alanine substitutions at either residue 70 or 219 (K70A and K219A, respectively), and one containing both amino acid substitutions simultaneously (K70AK219A).

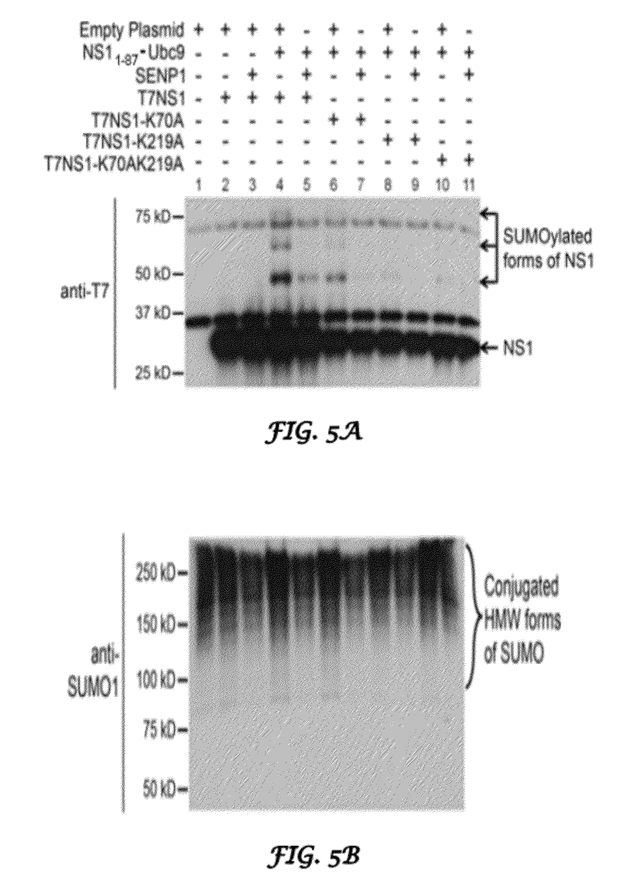

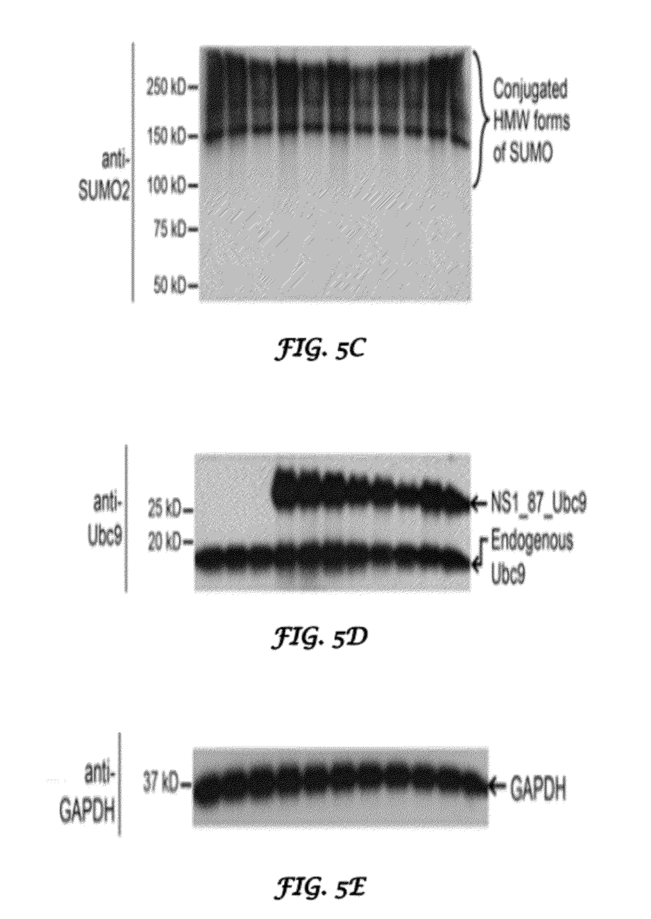

FIGS. 5A-5E shows that the ASUL activity mediated by NS1₁₋₈₇-Ubc9 is specific, i.e., that the ligase activity mediated by NS1₁₋₈₇-Ubc9 does not result in a global increase in cellular protein SUMOylation and that NS1₁₋₈₇-Ubc9 does not enhance the SUMOylation of a mutant form of the target protein lacking the specific lysine residues normally targeted by the SUMOylation system. In agreement with the data presented in FIG. 5, co-transfection with NS1₁₋₈₇-Ubc9 led to a dramatic increase in NS1 SUMOylation (FIG. 5A, lane 4), characterized by the appearance of three new bands whose molecular weight corresponded to the expected increase mediated by the addition of one, two, or three SUMO molecules. The ˜50 kD SUMOylated form was detected even in the presence of over-expressed SENP1 (FIG. 5A, lane 5). However, a dramatic decrease in the effect mediated by co-expression of NS1₁₋₈₇-Ubc9 was observed on the K70A and K219A NS1 mutants (FIG. 5A, lanes 6 and 8 respectively), and almost no SUMOylated forms were induced by NS1₁₋₈₇-Ubc9 on the K70AK219A double mutant form of NS1 (FIG. 5A, lane 10). Importantly, the anti-SUMO1 and anti-SUMO2 immunoblots demonstrated that SENP1 over-expression decreased global cellular SUMOylation, whereas over-expression of NS1₁₋₈₇-Ubc9 did not produce a detectable increase in global cellular SUMO1 or SUMO2 SUMOylation (FIGS. 5B and 5C). Altogether, the data presented demonstrates that the NS1₁₋₈₇-Ubc9 construct increased NS1 SUMOylation by enhancing the SUMO modification of the specific Lysine residues previously identified as the major SUMOylation sites in NS1. Therefore the ASUL activity associated to NS1₁₋₈₇-Ubc9 is highly specific, as initially intended.

The invention further describes two main potential applications for the technology described herein:

I. Characterizing the functional effects of SUMOylation on any given target: The overall technology described herein allows the rapid development of Artificial SUMO Ligases (ASULs) for any given SUMO target, provided that knowledge of the localization of the SUMOylation site and of specific protein interacting partners may exist. The development of such ASULs would facilitate the subsequent characterization of the effects mediated by SUMOylation of the specific target under study by providing conditions to specifically increase the SUMOylation of such target in the absence of detectable increases in global cellular SUMOylation. Importantly, in sharp contrast with the UFSD methodology described by Jacobs et al., since no modifications of the SUMO target under study are needed, this methodology will allow the characterization of the effects of SUMOylation under normal physiological levels of expression of the desired target.

II. Treatment of specific human diseases by enhancing protein solubility and decreasing protein aggregation: One of the most impressive properties mediated by SUMO is its ability to increase the solubility and decrease the aggregation of other proteins¹⁰. Decreased SUMOylation of the Amyloid Protein Precursor (APP) has been identified as a predisposing factor for the formation of Amyloid beta (Aβ) protein aggregates¹¹. These Aβ protein aggregates have been characterized as one of the causative mechanisms of Alzheimer's disease. Therefore, increasing APP SUMOylation has been postulated to constitute a potential molecular treatment for Alzheimer's disease¹². The development of an APP-specific ASUL could provide an effective tool for the molecular treatment of Alzheimer's disease by enhancing APP's SUMOylation. Similarly, a disease known as familial dilated cardiomyopathy has also been directly associated with decreases in the SUMOylation of a protein known to play key roles in nuclear structure and function, Lamin A¹³. The development of a Lamin A-specific ASUL could provide a tool for the molecular treatment of this disease. In general, as our knowledge of the molecular defects associated with different diseases continues to increase, it is likely that other diseases associated with defects in the SUMOylation of specific cellular proteins will be identified. The ASUL technology will provide a way to develop highly specific molecular treatments for such diseases.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,750,134: Nucleic Acids Encoding Microbial SUMO Protease Homologs.
1. Geiss-Friedlander R, Melchior F. Concepts in Sumoylation: A Decade On. Nat Rev Mol Cell Biol 2007; 8:947-56.
2. Dohmen R J. SUMO Protein Modification. Biochim Biophys Acta 2004; 1695:113-31.
3. Johnson E S. Protein Modification by SUMO. Annu Rev Biochem 2004; 73:355-82.
4. Niedenthal R. Ubc9 Fusion-Directed SUMOylation (UFDS). Biochem Soc Trans 2007; 35:1430-2.
5. Jakobs A, Himstedt F, Funk M, Korn B, Gaestel M, Niedenthal R. Ubc9 Fusion-Directed SUMOylation Identifies Constitutive and Inducible SUMOylation. Nucleic Acids Res 2007; 35:e109.
6. Jakobs A, Koehnke J, Himstedt F, et al. Ubc9 Fusion-Directed SUMOylation (UFDS): A Method to Analyze Function of Protein SUMOylation. Nat Methods 2007; 4:245-50.
7. Wang Y, Abu Irqeba A, Ayalew M, Suntay K. Sumoylation of Transcription Factor Tec1 Regulates Signaling of Mitogen-Activated Protein Kinase Pathways in Yeast. PLoS One 2009; 4:e7456.
8. Palvimo J J. PIAS Proteins as Regulators of Small Ubiquitin-Related Modifier (SUMO) Modifications and Transcription. Biochem Soc Trans 2007; 35:1405-8.
9. Pal S, Rosas J M, Rosas-Acosta G. Identification of the Non-Structural Influenza A Viral Protein NS1A as A bona fide Target of the Small Ubiquitin-like MOdifier by the Use of Dicistronic Expression Constructs. J Virol Methods 2009.
10. Panavas T, Sanders C, Butt T R. SUMO Fusion Technology for Enhanced Protein Production in Prokaryotic and Eukaryotic Expression Systems. Methods Mol Biol 2009; 497:303-17.
11. Zhang Y Q, Sarge K D. SUMOylation of Amyloid Precursor Protein Negatively Regulates Abeta Aggregate Levels. Biochem Biophys Res Commun 2008; 374:673-8.
12. Sarge K D, Park-Sarge O K. SUMOylation and Human Disease Pathogenesis. Trends Biochem Sci 2009; 34:200-5.
13. Zhang Y Q, Sarge K D. SUMOylation Regulates Lamin A Function and is Lost in Lamin A Mutants Associated with Familial Cardiomyopathies. J Cell Biol 2008; 182:35-9.

What is claimed is:

1. A fusion protein for increasing a rate, a specificity, or both of Small Ubiquitin-like Modifier attachment (SUMOylation) to a target protein comprising:
an interaction domain (ID) comprising a protein, a protein fragment, a peptide or combinations and modifications thereof, wherein the ID capable of interacting or binding to the target protein, a region or a site of the target protein undergoing the SUMOylation;
one or more conjugating enzymes or enzyme complexes, wherein the conjugating enzymes catalyze, modulate, or promote the SUMOylation in the target protein;
a Small Ubiquitin-like Modifier (SUMO) attached to the enzyme or the enzyme complex; and
a spacer, a linker, or a hinge region connecting the ID with the one or more enzymes or enzyme complexes.

2. The fusion protein of claim 1, wherein the target protein is a bacterial protein, a viral protein, a plant protein, an animal protein, a human protein, or combinations thereof.

3. The fusion protein of claim 1, wherein the conjugating enzyme comprises an ubiquitin-conjugating enzyme.

4. The fusion protein of claim 3, wherein the ubiquitin-conjugating enzyme is Ubc9.

5. The fusion protein of claim 1, wherein the fusion protein is activated by one or more SUMO activating enzymes.

6. The fusion protein of claim 5, wherein the SUMO activating enzyme is an E1 SUMO activating enzyme (SAE2/1).

7. The fusion protein of claim 1, wherein the enzyme or the enzyme complex may optionally comprise at least one of SUMO activating enzymes (E1), SUMO ligases (E3), or a protein inhibitor of activated Stat proteins (PIAS) comprising PIAS1 and PIASxβ, protein inhibitor of activated STAT proteins comprising Siz1 and Siz2/Nfi1, and Mms21.

8. The fusion protein of claim 1, wherein a length of the spacer is determined by both a size of the ID and a distance of the ID from a SUMOylation site in the target protein.

9. The fusion protein of claim 1, wherein the spacer orients the one or more enzymes or enzyme complexes with respect to the target protein or the SUMOylation site in the target protein.

10. A fusion protein (NS1₁₋₈₇-Ubc9) for increasing a rate, a specificity, or both of Small Ubiquitin-like Modifier attachment (SUMOylation) to an influenza A virus non-structural protein (NS1) comprising:
an interaction domain (ID) comprising amino acids 1-87 from a N-terminal region of the NS1 protein;
an ubiquitin-conjugating enzyme, wherein the ubiquitin-conjugating enzyme is Ubc9;
a Small Ubiquitin-like Modifier (SUMO) attached to the Ubc9; and
a spacer, a linker, or a hinge region connecting the ID with the Ubc9.

11. A method for increasing a rate, a specificity, or both of Small Ubiquitin-like Modifier attachment (SUMOylation) to a target protein in a human or an animal subject, a plant, or a cell comprising the step of injecting, introducing, or transfecting a fusion protein or one or more plasmids expressing the fusion protein, wherein the fusion protein comprises:
an interaction domain (ID) comprising a protein, a protein fragment, a peptide or combinations and modifications thereof, wherein the ID capable of interacting or binding to the target protein, a region, or a site of the target protein undergoing the SUMOylation;
one or more conjugating enzymes or enzyme complexes, wherein the conjugating enzymes catalyze, modulate, or promote the SUMOylation in the target protein;
a Small Ubiquitin-like Modifier (SUMO) attached to the enzyme or the enzyme complex; and
a spacer, a linker, or a hinge region connecting the ID with the one or more enzymes or enzyme complexes.

12. The method of claim 11, wherein the method further comprises the step of measuring a level of SUMOylation, an increase in a level of a SUMOylated target protein, or both in the human or the animal subject, the plant, or the cell prior to and after the injection, introduction, or transfection of the fusion protein or the one or more expression plasmids.

13. The method of claim 11, wherein the conjugating enzyme comprises an ubiquitin-conjugating enzyme.

14. The method of claim 13, wherein the ubiquitin-conjugating enzyme is Ubc9.

15. The method of claim 11, wherein the fusion protein is activated by one or more SUMO activating enzymes.

16. The method of claim 15, wherein the SUMO activating enzyme is an E1 SUMO activating enzyme (SAE2/1).

17. The method of claim 11, wherein the enzyme or the enzyme complex may optionally comprise one or more SUMO activating enzymes (E1), SUMO ligases (E3), or a protein inhibitor of activated Stat proteins (PIAS) comprising PIAS1 and PIASxβ, protein inhibitor of activated STAT proteins comprising Siz1 and Siz2/Nfi1, and Mms21.

18. The method of claim 11, wherein a length of the spacer is determined by both a size of the ID and a distance of the ID from a SUMOylation site in the target protein.

19. The method of claim 11, wherein the spacer orients the one or more enzymes or enzyme complexes with respect to the target protein or the SUMOylation site in the target protein.

20. A method for increasing a rate, a specificity or both of Small Ubiquitin-like Modifier attachment (SUMOylation) to an influenza A virus non-structural protein (NS1) in a human or animal subject or a cell comprising the steps of injecting, introducing or transfecting a fusion protein (NS1₁₋₈₇-Ubc9), one or more plasmids expressing the fusion protein, wherein the fusion protein comprises:
an interaction domain (ID) comprising amino acids 1-87 from a N-terminal region of the NS1 protein;
an ubiquitin-conjugating enzyme, wherein the ubiquitin-conjugating enzyme is Ubc9;
a Small Ubiquitin-like Modifier (SUMO) attached to the Ubc9; and
a spacer, a linker, or a hinge region connecting the ID with the Ubc9.

21. The method of claim 20, wherein the method further comprises the step of measuring a level of SUMOylation, an increase in a level of a SUMOylated NS1 protein, or both in the human or animal subject or the cell prior to and after the introduction or transfection of the one or more expression plasmids.

22. A method of characterizing a target protein, studying a structure, a function, or any combinations thereof comprising the steps of:
promoting, increasing a rate, a specificity, or combinations thereof of Small Ubiquitin-like Modifier attachment (SUMOylation) to the target protein in a human or animal subject, a plant, or a cell;
providing a fusion protein comprising:
an interaction domain (ID) comprising a protein, a protein fragment, a peptide, or combinations and modifications thereof, wherein the ID capable of interacting or binding to the target protein, a region or a site of the target protein undergoing the SUMOylation;
one or more conjugating enzymes or enzyme complexes, wherein the conjugating enzymes catalyze, modulate or promote the SUMOylation in the target protein;
a Small Ubiquitin-like Modifier (SUMO) attached to the enzyme or the enzyme complex; and
a spacer, a linker or a hinge region connecting the ID with the one or more enzymes or enzyme complexes;
introducing or transfecting the fusion protein or one or more plasmids expressing the fusion protein in the human or animal subject, the plant, or the cell;
measuring a level of SUMOylation, an increase in a level of a SUMOylated target protein, or both in the human or animal subject, the plant, or the cell; and
characterizing the SUMOylated target protein, studying the structure, the function, or any combinations thereof by using one or more analytical, structural, or functional techniques.

23. A method for treating Alzheimer's disease (AD) in a human subject by increasing a rate, a specificity or combinations thereof of Small Ubiquitin-like Modifier attachment (SUMOylation) to one or more amyloid precursor proteins (APP), wherein the SUMOylation increases a solubility or decreasing an aggregation or both, comprising the steps of:
identifying the subject in need for treatment against the AD; and
administering a therapeutically effective amount of a fusion protein comprising:
an interaction domain (ID) comprising a protein, a protein fragment, a peptide, or combinations and modifications thereof, wherein the ID capable of interacting or binding to the APP, a region, or a site of the APP undergoing the SUMOylation;
one or more conjugating enzymes or enzyme complexes, wherein the conjugating enzymes catalyze, modulate, or promote the SUMOylation in the APP;
a Small Ubiquitin-like Modifier (SUMO) attached to the enzyme or the enzyme complex; and
a spacer, a linker, or a hinge region connecting the ID with the one or more enzymes or enzyme complexes.

24. The method of claim 23, further comprising the step of monitoring a progression of the treatment by measuring a change in the solubility, the aggregation, or both of the one or more APP prior to or after the administration of the fusion protein.

25. The method of claim 23, wherein the fusion protein is administered parenterally.

26. A method for treating one or more diseases associated with defects in the Small Ubiquitin-like Modifier attachment SUMOylation of specific cellular proteins in a human subject by increasing a rate, a specificity, or combinations thereof of (SUMOylation) to one or more cellular proteins comprising the steps of:
identifying the subject in need for treatment against the one or more diseases; and
administering a therapeutically effective amount of a fusion protein comprising an interaction domain (ID) comprising a protein, a protein fragment, a peptide, or combinations and modifications thereof, wherein the ID capable of interacting or binding to the cellular protein, a region, or a site of the cellular protein undergoing the SUMOylation;
one or more conjugating enzymes or enzyme complexes, wherein the conjugating enzymes catalyze, modulate, or promote the SUMOylation in the cellular protein;
a Small Ubiquitin-like Modifier (SUMO) attached to the enzyme or the enzyme complex; and
a spacer, a linker, or a hinge region connecting the ID with the one or more enzymes or enzyme complexes.

27. The method of claim 26, further comprising the step of monitoring a progression of the treatment by measuring a level of SUMOylation, an increase in a level of a SUMOylated cellular protein, or both in the human subject prior to and after the administration of the fusion protein.

28. The method of claim 26, wherein the disease is familial dilated cardiomyopathy.

29. The method of claim 26, wherein the cellular protein is Lamin A.
